(19) United States
(12) Patent Application Publication
(10) Pub. No.: US 2024/0166799 A1
(43) Pub. Date: May 23, 2024

(54) THERMALLY REACTIVE IMIDAZOLE-BASED LATENT CURING AGENT AND METHOD OF PREPARING THE SAME

(71) Applicant: KOREA INSTITUTE OF SCIENCE AND TECHNOLOGY, Seoul (KR)

(72) Inventors: Jaewoo KIM, Seoul (KR); Sungmin JUNG, Wanju-gun (KR); Yong Seok CHOI, Wanju-gun (KR); Jong Hyuk PARK, Seoul (KR); Min PARK, Wanju-gun (KR); Jun Woo JEON, Wanju-gun (KR); Yong Chae JUNG, Wanju-gun (KR); YOONSANG KIM, Wanju-gun (KR); Han Gyeol JANG, Wanju-gun (KR); Jun Young JO, Wanju-gun (KR)

(21) Appl. No.: 18/066,389

(22) Filed: Dec. 15, 2022

(30) Foreign Application Priority Data
Oct. 28, 2022 (KR) 10-2022-0141344

(51) Int. Cl. C08G 59/40; C07D 233/58

(52) U.S. Cl. CPC C08G 59/4042; C07D 233/58

(57) ABSTRACT

The present exemplary embodiments may provide a latent curing agent including an imidazole-based compound protected by a Diels-Alder reaction between a diene and a dienophile.

### CROSS-REFERENCE TO RELATED APPLICATION

This application claims priority to and the benefit of Korean Patent Application No. 10-2022-0141344 filed in the Korean Intellectual Property Office on Oct. 28, 2022, the entire contents of which are incorporated herein by reference.

### BACKGROUND OF THE INVENTION

### (a) Field of the Invention

The present exemplary embodiments relate to an imidazole-based latent curing agent and a method of preparing the same, and more particularly, to a thermally reactive imidazole-based latent curing agent protected by a Diels-Alder reaction between a diene and a dienophile and a method of preparing the same.

### (b) Description of the Related Art

Since an epoxy has excellent properties of mechanical strength, heat resistance, and chemical resistance, it is being widely used in various fields such as adhesives, coating, and composites. Since the properties of the epoxy is greatly different depending on the type of curing agents, various curing agents such as phenol, anhydrous acetic acid, carboxylic acid, amine, and imidazole are used depending on the use. Among these, since an imidazole curing agent has high reactivity with an epoxy resin, it causes a rapid curing reaction with a very small amount, thereby improving cost reduction of a process and productivity. Therefore, it is being widely used in the fields such as aerospace, automobiles, electrical/electronic, and architecture.

A one-component epoxy system is supplied in a previously combined state of an epoxy resin and a curing agent. Therefore, it does not need to be weighed, has excellent convenience, and has constant performance. In this respect, the use of the one-component epoxy in an actual process becomes increasingly common. However, since an imidazole has high reactivity, it is very difficult to secure storage stability when previously combined. Therefore, a study to develop a curing agent which secures storage stability by chemical or physical modification and initiates a reaction under specific conditions is being actively conducted.

Up to date, in order to improve the storage stability of an imidazole, development of a microcapsule having a size of 4 to 30 μm having high stability by shelling the imidazole with a polymer and an imidazole-metal ion complex curing agent having reactivity controlled by a coordination bond formed between a transition metal and the imidazole has been carried out. In addition, development of an imidazole derivative having reactivity adjusted by introducing various functional groups by a chemical reaction is also being carried out. However, latent curing agents which have been developed so far are difficult to be mass produced, are complicatedly synthesized, and have low resistance to an organic solvent.

Therefore, development of a new latent curing agent which may overcome the drawbacks is required.

The above information disclosed in this Background section is only for enhancement of understanding of the background of the invention, and therefore it may contain information that does not form the prior art that is already known in this country to a person of ordinary skill in the art.

### SUMMARY OF THE INVENTION

The present invention has been made in an effort to provide a thermally reactive imidazole-based latent curing agent having an advantage of excellent storage stability, and a method of preparing a latent curing agent by a simple preparation process.

An exemplary embodiment of the present invention provides a latent curing agent including an imidazole-based compound protected by a Diels-Alder reaction between a diene and a dienophile.

The imidazole-based compound may be represented by the following Chemical Formula 1:

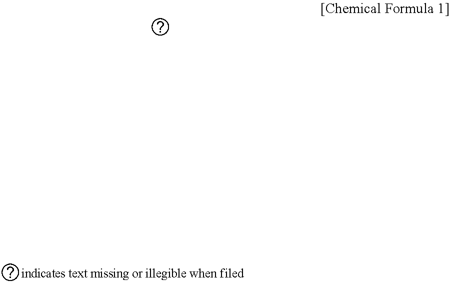

wherein A₁ to A₄ are independently of one another a hydrogen atom, an alkyl group having 1 to 15 carbon atoms, a phenyl group, or a heterocyclic compound.

The diene may include one or more diene functional groups of the following Chemical Formula 2-1 to Chemical Formula 2-14 or derivatives thereof:

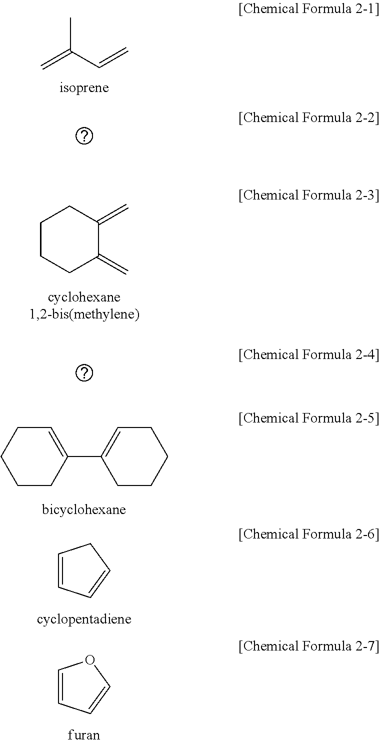

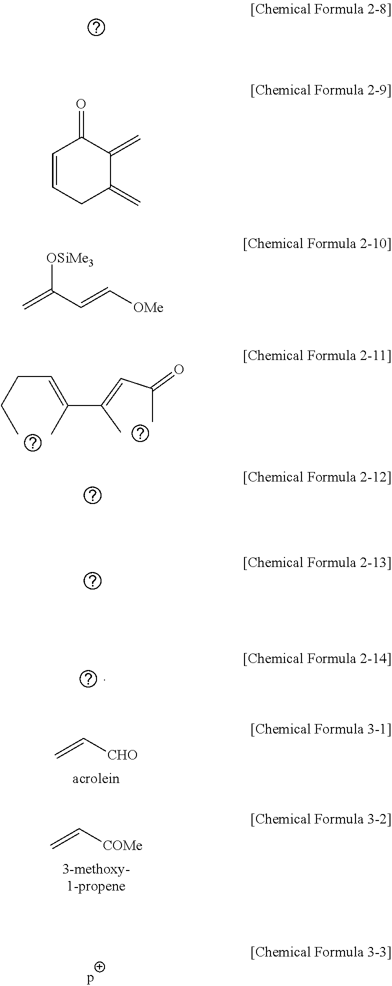

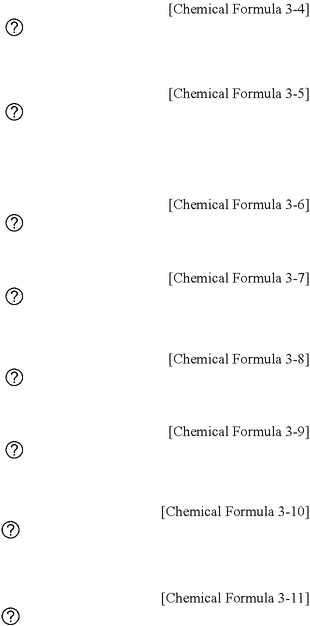

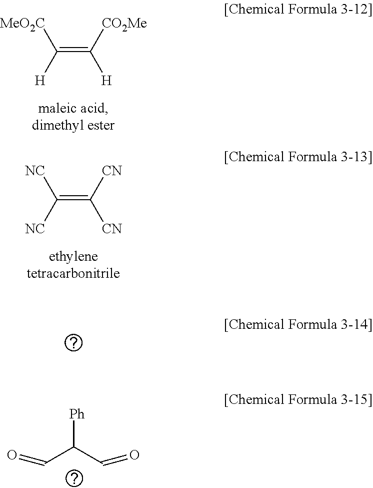 [SMILES: COC(=O)/C=C\C(=O)OC ; N#CC(C#N)=C(C#N)C#N ; O=CC(C=O)c1ccccc1]

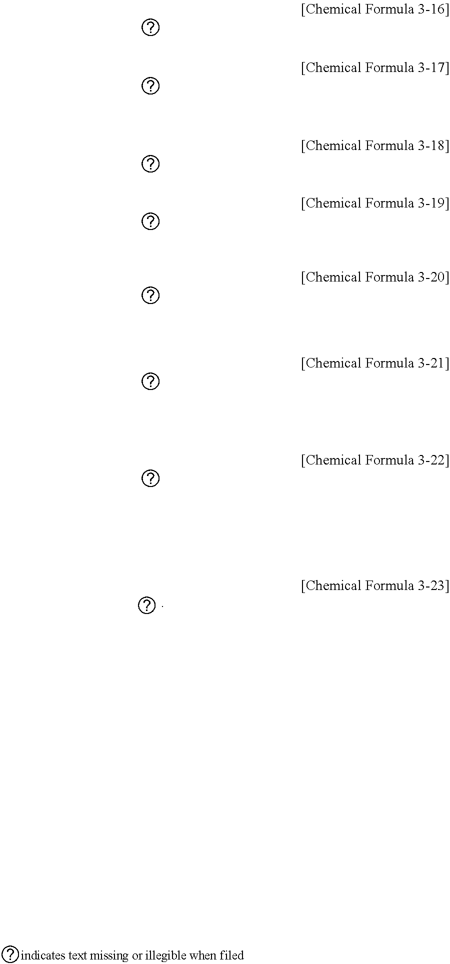

The dienophile may include one or more dienophile functional groups of the following Chemical Formula 3-1 to Chemical Formula 3-23 or derivatives thereof:

[symbol]

The latent curing agent may start curing in a temperature range of higher than 80° C. and 200° C. or lower, and may be protected by a Diels-Alder reaction between a diene and a dienophile at a temperature in a range of 20° C. to 80° C.

The latent curing agent may have an average particle size (D50) in a range of 10 nm to 100 mm.

The imidazole-based compound may be included in a range of 20 wt % to 90 wt % of the latent curing agent by weight.

In addition, a diene functional group included in the diene and a dienophile functional group included in the dienophile may be included in a range of 0.2:1 to 5:1 based on moles.

Another embodiment of the present invention provides a method of preparing a latent curing agent including: mixing a diene, a dienophile, and an imidazole-based compound to obtain a first mixed solution; mixing the first mixed solution with a surfactant to obtain a second mixed solution; and post-treating the second mixed solution to obtain a latent curing agent, wherein the obtaining of a second mixed solution may be performed in a temperature range of 0° C. to 150° C.

In the method of preparing a latent curing agent, the imidazole-based compound may be represented by Chemical Formula 1:

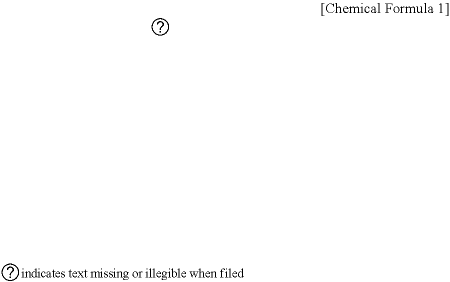

wherein A₁ to A₄ are independently of one another a hydrogen atom, an alkyl group having 1 to 15 carbon atoms, a phenyl group, or a heterocyclic compound.

In the method of preparing a latent curing agent, the imidazole-based compound may be represented by Chemical Formula 1.

In the method of preparing a latent curing agent, the diene may include one or more diene functional groups of the following Chemical Formula 2-1 to Chemical Formula 2-14 or derivatives thereof, and the dienophile may include one or more dienophile functional groups of the following Chemical Formula 3-1 to Chemical Formula 3-23 or derivatives thereof.

The mixing of a diene, a dienophile, and an imidazole-based compound to obtain a first mixed solution may be mixing at a ratio of 0.2:1 to 5:1 based on moles of the diene functional group of the diene and the dienophile functional group of the dienophile.

In the mixing of a diene, a dienophile, and an imidazole-based compound to obtain a first mixed solution, the imidazole-based compound may be included in a range of 20 wt % to 90 wt %, with respect to the total weight of the diene, the dienophile, and the imidazole-based compound.

The thermally reactive imidazole-based latent curing agent according to an exemplary embodiment of the present invention has excellent storage stability.

The method of preparing an imidazole-based latent curing agent according to another exemplary embodiment of the present invention has a simple preparation process, is economical, and has a high reaction rate.

### BRIEF DESCRIPTION OF THE DRAWINGS

FIG. 1 is a schematic diagram of a preparation process of a latent curing agent according to an exemplary embodiment.

FIG. 2 is FE-SEM images of curing agents according to Examples 1 to 3 and Comparative Example 1.

FIG. 3 shows particle size analysis results of the curing agents according to Examples 1 to 3 and Comparative Example 1.

FIG. 4 is FE-SEM images of the curing agents according to Examples 1 to 3 and Comparative Example 1 after a heat treatment (after causing a retro-Diels-Alder reaction) of the curing agents.

FIG. 5A shows DSC analysis results of mixtures of a curing agent and an epoxy according to Experimental Examples 1 to 3 and Comparative Example 2, and FIG. 5B shows DSC analysis results of the curing agents themselves according to Examples 1 to 3 and Comparative Example 1.

FIG. 6 shows DSC analysis result of the mixtures of a curing agent and an epoxy according to Experimental Examples 1 to 3 immediately after preparation and after storage for 30 days of the mixtures.

FIG. 7 shows a specification of specimens prepared according to Experimental Examples 1 to 3 and Comparative Experimental Example 2.

FIG. 8 shows tensile strength test results of the specimens prepared according to Experimental Examples 1 to 3 and Comparative Experimental Example 2.

FIG. 9 shows shear strength test results of the specimens prepared according to Experimental Examples 1 to 3 and Comparative Experimental Example 2.

### DETAILED DESCRIPTION OF THE EMBODIMENTS

The terminology used herein is only for mentioning a certain example, and is not intended to limit the present invention. Singular forms used herein also include plural forms unless otherwise stated clearly to the contrary. The meaning of “comprising” used in the specification is embodying certain characteristics, regions, integers, steps, operations, elements, and/or components, but is not excluding the presence or addition of other characteristics, regions, integers, steps, operations, elements, and/or components.

Though not defined otherwise, all terms including technical terms and scientific terms used herein have the same meaning as commonly understood by a person with ordinary skill in the art to which the present invention pertain. Terms defined in commonly used dictionaries are further interpreted as having a meaning consistent with the related technical literatures and the currently disclosed description, and unless otherwise defined, they are not interpreted as having an ideal or very formal meaning.

In addition, unless particularly mentioned, % refers to wt % or mass %, and 1 ppm is 0.0001 wt % or 0.0001 mass %.

The terms such as first, second, and third are used for describing various parts, components, areas, layers, and/or sections, but are not limited thereto. These terms are used only for distinguishing one part, component, area, layer, or section from other parts, components, areas, layers, or sections. Therefore, a first component, part, area, layer, or section described below may be mentioned as a second component, part, area, layer, or section without departing from the scope of the present invention.

Hereinafter, exemplary embodiments of the present invention will be described in detail. However, these are presented as an example and the present invention is not limited thereto, and the present invention only defined by the scope of the claims described later.

An exemplary embodiment of the present invention may provide a method of preparing a latent curing agent.

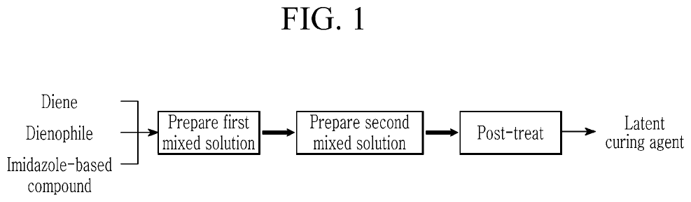

FIG. 1 is a schematic diagram of a preparation process of a latent curing agent according to an exemplary embodiment.

Referring to FIG. 1, the method of preparing a latent curing agent according to the present invention may include: using a diene, a dienophile, and an imidazole-based compound to prepare a first mixed solution; dispersing the first mixed solution in a surfactant solution and performing stirring to prepare a second mixed solution including a latent curing agent in a solid material form; and post-treating the second mixed solution.

First, a diene including a diene functional group, a dienophile including a dienophile functional group, and an imidazole-based compound may be dissolved in an organic solvent to prepare a first mixed solution.

The thus-prepared first mixed solution may be dispersed in an excessive amount of a surfactant solution to form a suspension, that is, an emulsion. The suspension may be stirred in a temperature range of 0° C. to 150° C., specifically 20° C. to 80° C. at a stirring speed of 100 rpm to 100000 rpm for 1 hour or more to prepare a second mixed solution including a solid latent curing agent having a smooth surface and a spherical shape.

The post-treating of a second mixed solution including a solid latent curing agent may include removing the organic solvent at room temperature, removing the surfactant using an excessive amount of water and a centrifuge, and removing water using a lyophilizer.

The latent curing agent according to another exemplary embodiment of the present invention may be an imidazole-based latent curing agent, and specifically, may be a latent curing agent including an imidazole-based compound protected by a Diels-Alder reaction between a diene and a dienophile.

The imidazole-based compound may be represented by the following Chemical Formula 1:

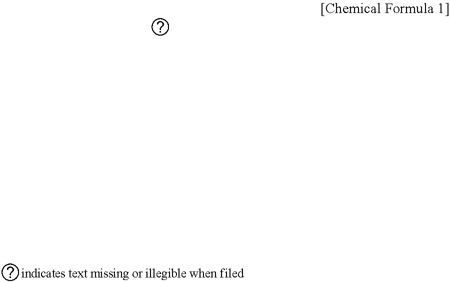

wherein A₁ to A₄ are independently of one another a hydrogen atom, an alkyl group having 1 to 15 carbon atoms, a phenyl group, or a heterocyclic compound.

The diene may include one or more diene functional groups of the following Chemical Formula 2-1 to Chemical Formula 2-14 or derivatives thereof. Here, the functional group is a simple example, and is not limited only to the functional groups represented by Chemical Formula 2-1 to Chemical Formula 2-14, in the present application:

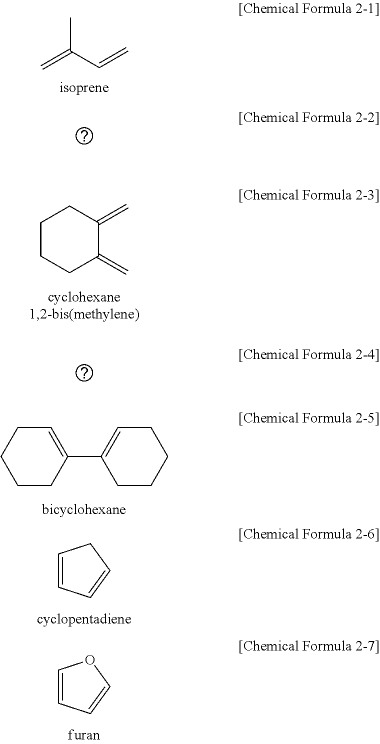

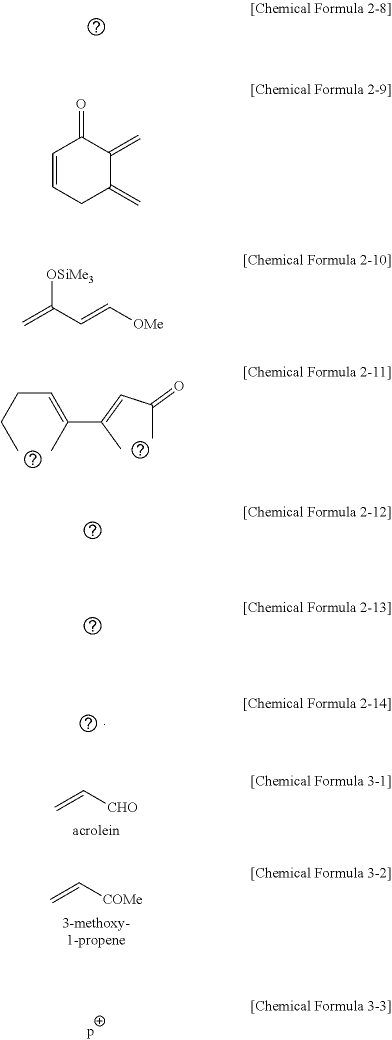

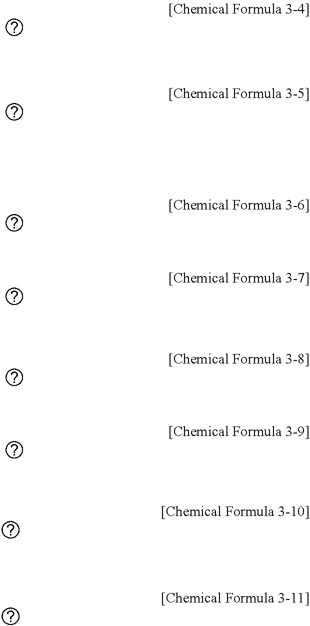

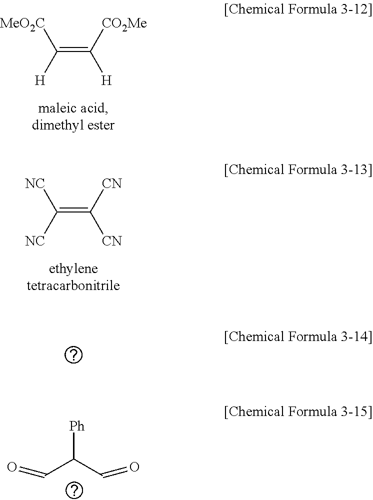 [SMILES: COC(=O)/C=C\C(=O)OC ; N#CC(C#N)=C(C#N)C#N ; O=CC(C=O)c1ccccc1]

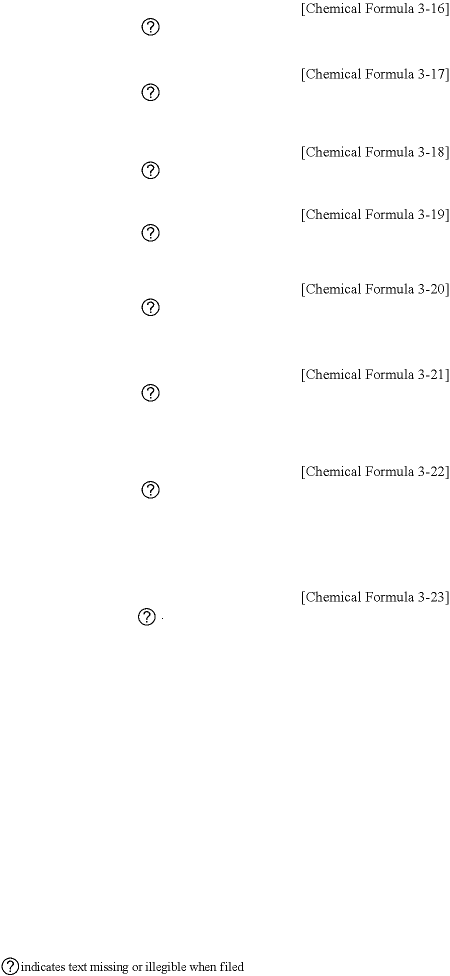

The dienophile may include one or more dienophile functional groups of the following Chemical Formula 3-1 to Chemical Formula 3-23 or derivatives thereof. Here, the functional group is a simple example, and is not limited only to the functional groups represented by Chemical Formula 2-1 to Chemical Formula 2-14, in the present application:

[symbol]

One or more diene functional groups included in the diene and one or more dienophile functional groups included in the dienophile may cause a Diels-Alder reaction at a temperature in a range of 150° C. or lower, specifically 20° C. to 120° C., and more specifically 20° C. to 80° C. to protect a latent curing agent and improve storage stability.

Meanwhile, as the temperature is raised, a retro-Diels-Alder reaction which is a decomposition reaction of the Diels-Alder reaction by the diene functional group and the dienophile functional group occurs, so that the latent curing agent starts curing. The temperature at which the retro-Diels-Alder reaction occurs may be in a range of 60° C. to 200° C., specifically higher than 80° C. and 200° C. or lower, and more specifically 150° C. to 200° C.

The imidazole-based compound may be included in a range of 20 wt % to 90 wt %, specifically 30 wt % to 60 wt % of the latent curing agent by weight. When the imidazole-based compound is included at a higher weight, reactivity is increased so that storage stability is not secured, and when included at a lower weight, the gravity of the curing agent is small, so that curing does not proceed well.

The diene functional group included in the diene and the dienophile functional group included in the dienophile may be included in a range of 0.2:1 to 5:1, specifically at 1:1, based on moles. When the diene functional group included in the diene and the dienophile functional group included in the dienophile are included at the above mole ratio, the Diels-Alder reaction inside the latent curing agent occurs effectively, which is advantageous for stably storing the latent curing agent.

Here, the latent curing agent may be microbeads, and specifically, may be spherical particles. The latent curing agent may have an average particle size (D50) in a range of 10 nm to 100 mm range, specifically in a range of 0.1 μm to 10 μm. When the average particle size (D50) of the latent curing agent is within the range, the latent curing agent is uniformly mixed with an epoxy resin and has improved dispersion stability to maintain the quality and performance of a cured epoxy.

### EXAMPLE 1

450 mg of a tetrafunctional furan (TFu) (1.9 mmol of a furan group), 350 mg of bismaleimide (1.9 mmol of a maleimide group), and 800 mg of benzyl 2-methylimidazole (BMI) were dissolved in 1.2 mL of dichloromethane (DCM) to prepare a first solution at room temperature. At this time, a mass ratio of BMI, TFu, and BM was BMI/(TFu+BM)=1. The thus-prepared first solution was mixed with 30 mL of a 5 wt % polyvinyl alcohol (PVA) aqueous solution and then a homogenizer was used at 6000 rpm for 5 minutes to prepare a suspension. The suspension was heated at 40° C. for 1 hour. The organic solvent was removed at room temperature for 2 hours to 3 hours and diluted in sufficient water, and then a centrifuge was used to remove all of the surfactant. Finally, a lyophilizer was used for 24 hours to 48 hours to completely remove remaining water to obtain a latent curing agent.

### EXAMPLE 2

A latent curing agent was prepared in the same manner as in Example 1, except that the mass ratio of BMI, TFu, and BM was BMI/(TFu+BM)=1.5.

### EXAMPLE 3

A latent curing agent was prepared in the same manner as in Example 1, except that the mass ratio of BMI, TFu, and BM was BMI/(TFu+BM)=2.

### Comparative Example 1

A control group sample was prepared in the same manner as in Example 1, except that BMI was not mixed therein. Since it did not include BMI, it was not able to be used as a curing agent.

### Comparative Example 2

BMI used in Example 1 was used as a curing agent.

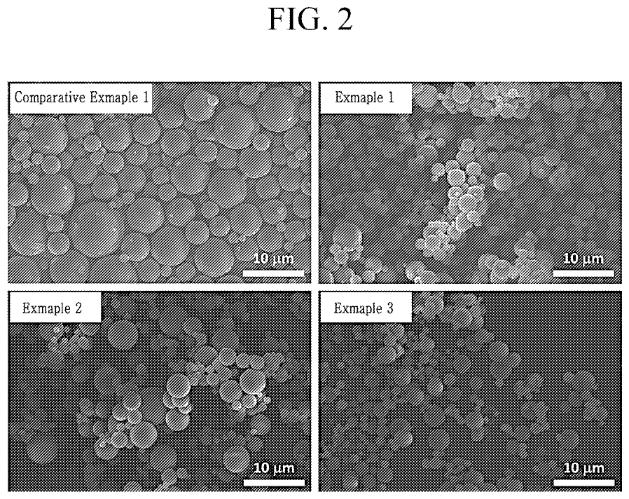

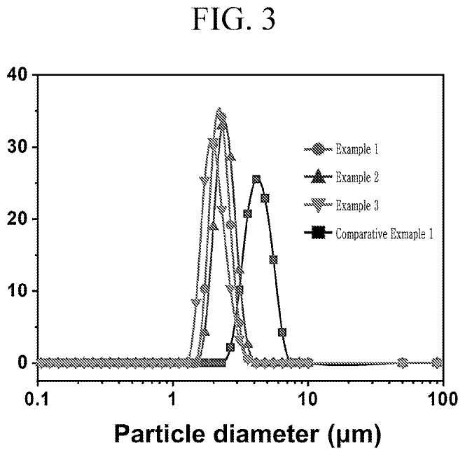

FIG. 2 is FE-SEM images of the latent curing agents according to Examples 1 to 3 and Comparative Example 1, and FIG. 3 shows particle size analysis results of the latent curing agents according to Examples 1 to 3 and Comparative Example 1.

Referring to FIG. 2, it may be confirmed that the latent curing agents prepared according to Examples 1 to 3 and Comparative Example 1 of the present invention have a spherical particle shape, and the spherical particle shape of the latent curing agent prepared according to Comparative Example 1 had a larger particle size than those of the latent curing agents prepared according to Examples 1 to 3.

Referring to FIG. 3, it was confirmed that the average particle size (D50) of the latent curing agent according to Comparative Example 1 of the present invention was 4.7 μm, and the average particle size (D50) of the latent curing agents according to Examples 1 to 3 was 2.4 μm, 2.6 μm, and 2.2 μm, respectively.

The following Table 1 shows the results of TGA analysis and DSC analysis of the latent curing agents and BMI according to Examples 1 to 3 and Comparative Example 1.

The BMI content included in the latent curing agents and the decomposition temperature thereof were confirmed by thermogravimetric analysis (TGA), and the curing start temperature, the curing peak temperature, and the storage stability of the latent curing agents were analyzed by differential scanning calorimetry (DSC) analysis.

TABLE 1

| | TGA | | DSC | | |
|---|---|---|---|---|---|
| | BMI | | | | Storage |
| | content | T_{d} | Tₒₙ | Tₘₐₓ | stability |
| | (%) | (° C.) | (° C.) | (° C.) | (day) |
| Pure BMI | 100 | 150.6 | 118.4 | 135.4 | <2 |
| Comparative Example 1 | 0 | 300.1 | — | — | — |
| Example 1 | 10 | 205.0 | 134.3 | 145.2 | >30 |
| Example 2 | 13 | 196.9 | 133.5 | 146.0 | >30 |
| Example 3 | 17 | 187.2 | 126.8 | 148.8 | >30 |

In Table 1, T_{d} refers to a temperature at which a weight of 5 wt % was decreased, Tₒₙ refers to a curing start temperature, and Tₘₐₓ refers to a curing peak temperature.

### Experimental Example 1

The latent curing agent prepared in Example 1 and an epoxy resin (YDF-170) were mixed at a weight ratio of 50:100 to prepare a mixture of a curing agent and an epoxy. The BMI content in the mixture of the latent curing agent and the epoxy resin (YDF-170) by weight was 5 wt %.

### Experimental Example 2

The latent curing agent prepared in Example 2 and an epoxy resin (YDF-170) were mixed at a weight ratio of 38.5:100 to prepare a mixture of a curing agent and an epoxy. The BMI content in the mixture of the latent curing agent and the epoxy resin (YDF-170) by weight was 5 wt %.

### Experimental Example 3

The latent curing agent prepared in Example 3 and an epoxy resin (YDF-170) were mixed at a weight ratio of 29.4:100 to prepare a mixed material of a curing agent and an epoxy. The BMI content in the mixture of the latent curing agent and the epoxy resin (YDF-170) by weight was 5 wt %.

### Comparative Experimental Example 1

The curing agent prepared in Comparative Example 1 and an epoxy resin (YDF-170) were mixed to prepare a mixture of a curing agent and an epoxy. At this time, the BMI content in the entire mixture was 5 wt %.

### Comparative Experimental Example 2

The curing agent prepared in Comparative Example 2 and an epoxy resin (YDF-170) were mixed at a weight ratio of 5:100 to prepare a mixture of a curing agent and an epoxy. At this time, the BMI content in the entire mixture was 5 wt %.

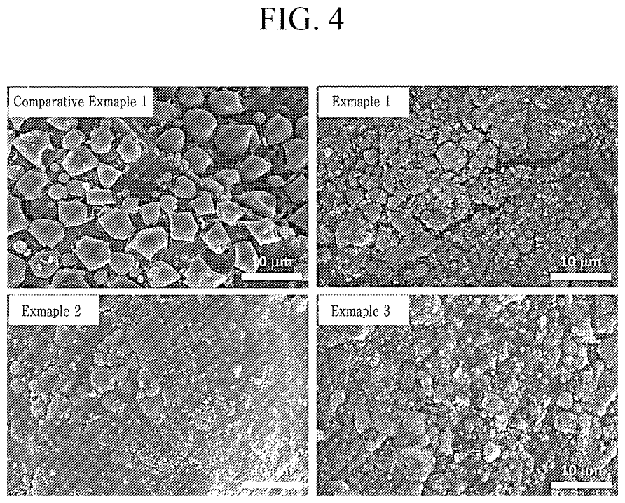

FIG. 4 is FE-SEM images of the curing agents according to Experimental Examples 1 to 3 and Comparative Experimental Example 1 after a heat treatment of the curing agents. Specifically, FE-SEM images after a heat treatment at 150° C. for 2 hours are shown.

Referring to FIG. 4, it may be confirmed in Experimental Examples 1 to 3 and Comparative Experimental Example 1 that a retro-Diels-Alder reaction occurs during the heat treatment at 150° C. for 2 hours, so that the spherical shape of the particles of FIG. 2 was not maintained.

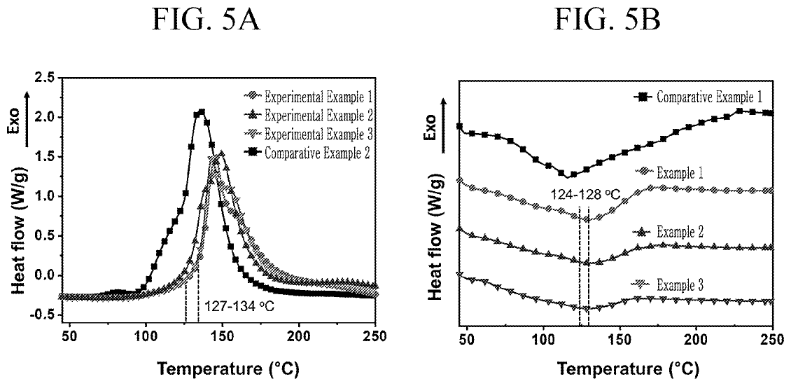

FIGS. 5A and 5B show the DSC analysis results according to Experimental Examples 1 to 3 and Comparative Experimental Example 2. Specifically, FIG. 5A shows the DSC analysis results of the curing reaction, and FIG. 5B shows the DSC analysis results of the retro-Diels-Alder reaction.

Referring to FIGS. 5A and 5B, it may be confirmed that when the latent curing agents prepared according to Examples 1 to 3 were applied, an exothermic area was decreased and both the curing start temperature and the curing peak temperature were raised. The decreased exothermic area was due to exothermic reaction offsetting by the endothermic reaction of the retro-Diels-Alder. Further, the raised curing start temperature and curing peak temperature were due to the reactivity inhibition by the operation of physical and chemical protections by a crosslinked network formed by the Diels-Alder reaction. It may be confirmed that only when a temperature at which the network decomposition (the retro-Diels-Alder reaction of (FIG. 5B) reached its peak was reached, the protection operation was released, and as a result, BMI was exposed to start the curing reaction with an epoxy resin.

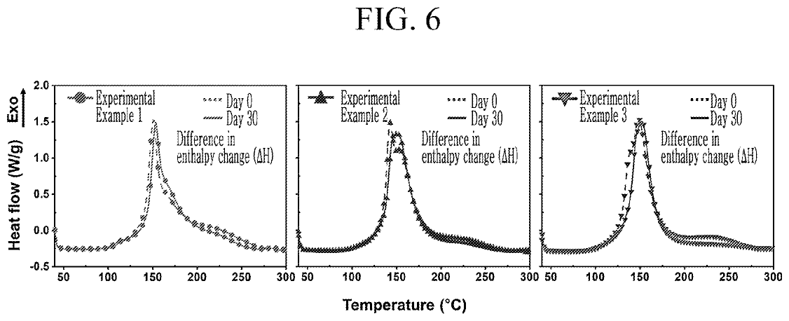

FIG. 6 shows the DSC analysis results of the mixtures of the curing agent and an epoxy according to Experimental Examples 1 to 3 immediately after preparation and after storage for 30 days of the mixtures. Specifically, (a), (b) and (c) are the results from Experimental Examples 1, 2, and 3, respectively. The DSC analysis results immediately after preparing the mixture of the curing agent and an epoxy and the DSC analysis results after storing the prepared mixture of the curing agent and an epoxy for 30 days under the conditions of a temperature of 20° C. are shown.

Referring to FIG. 6, it may be confirmed that a similar exothermic area was observed even after 30 days as compared with the exothermic area at the start day of the storage stability experiment, and the curing start/highest temperature was hardly changed. In addition, it was confirmed that a difference in an exothermic area ΔHₑₓₒ value was 5% or less even after storage for 30 days, and this proved that the storage stability of the Diels-Alder latent curing agent was excellent.

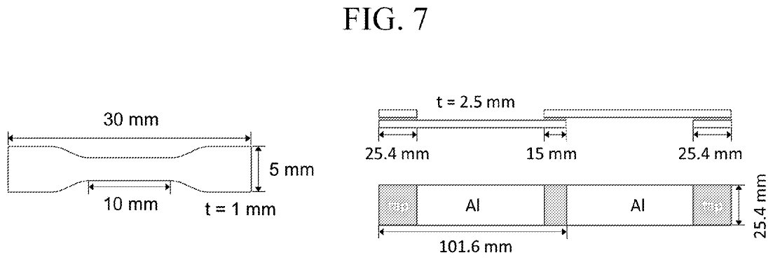

Curing agent-epoxy specimens were prepared using the mixtures of the curing agent and an epoxy according to Experimental Examples 1 to 3 and Comparative Example 2, and a mechanical strength performance test was performed. In the case of a tensile specimen, first, the mixture of the curing agent and an epoxy were poured into a silicon mold of 1×35×35 mm and cured at 150° C. for 2 hours. The prepared rectangular specimen was cut into a dog-bone shape of (a) of FIG. 7 using a laser cutting system, and then was used in the tensile test. As seen from (b) of FIG. 7, the tensile specimen was cured under the same conditions as in the preparation of the tensile strength specimen, after applying 30 mg of the mixture of the curing agent and an epoxy to an adhesive surface of an aluminum coupon (2.5×15×25.4 mm). The tensile and shear experiments were performed at speeds of 5 mm/min and 13 mm/min, through a universal material testing machine (Instron 5567, Instron Corp. USA) equipped with load cells of 2 kN and 30 kN capacities, respectively.

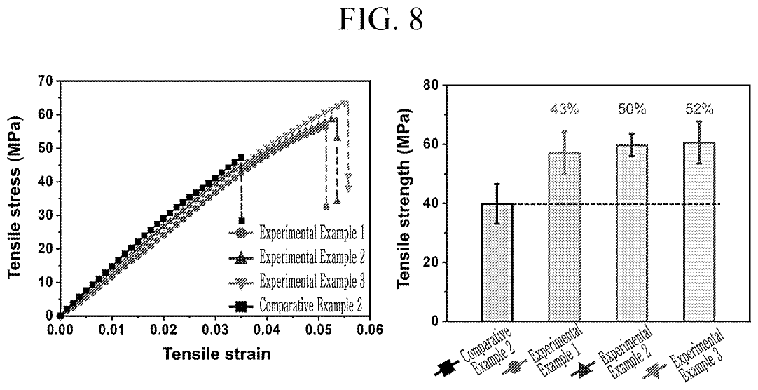

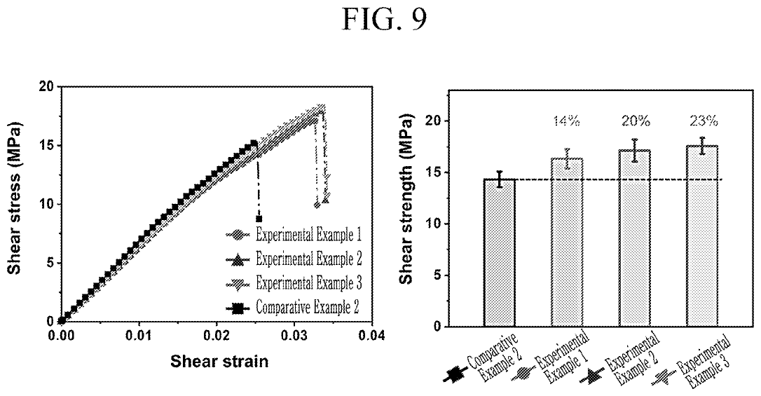

FIG. 8 shows the tensile strength test results of the specimens prepared according to Experimental Examples 1 to 3 and Comparative Experimental Example 2, and FIG. 8 shows the shear strength test results of the specimens prepared according to Experimental Examples 1 to 3 and Comparative Experimental Example 2. Specifically, in FIG. 8, (a) shows the tensile stress depending on a tensile strain, and (b) shows the tensile strength of each specimen. In addition, in FIG. 9, (a) shows the shear stress depending on a shear strain, and (b) shows the shear strength of each specimen.

Referring to FIG. 8, in the tensile strength test, all of the curing agent-epoxy specimens prepared using the mixtures of the curing agent and an epoxy according to Experimental Examples 1 to 3 and Comparative Experimental Example 2 had a reduced slope while showing viscoelastic behavior after passing through a first linear elastic section, and were finally broken. The tensile strain of the specimens prepared according to Experimental Examples 1 to 3 was increased by 49% to 61% as compared with the specimen according to Comparative Experimental Example 2, and the tensile strength thereof was increased by 43% to 52%.

Referring to FIG. 9, it was confirmed that the shear strength test results were similar to the tensile strength test results. In addition, the shear strain of the specimens prepared according to Experimental Examples 1 to 3 was increased by 23% to 31% as compared with the specimen according to Comparative Experimental Example 2, and the shear strength thereof was increased by 14% to 23%.

The present invention is not limited to the exemplary embodiments, but may be produced in various forms different from each other. A person with ordinary skill in the art to which the present invention pertains will understand that the present invention may be carried out in other specific forms without changing the spirit or the essential feature of the present invention. Therefore, the exemplary embodiments described above should be understood to be illustrative in all respects, and not to be restrictive.

What is claimed is:

1. A latent curing agent comprising:
an imidazole-based compound protected by a Diels-Alder reaction between a diene and a dienophile.

2. The latent curing agent of claim 1, wherein:
the imidazole-based compound is represented by the following Chemical Formula 1: 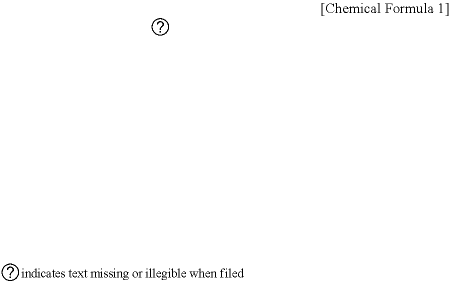
wherein A₁ to A₄ are independently of one another a hydrogen atom, an alkyl group having 1 to 15 carbon atoms, a phenyl group, or a heterocyclic compound.

3. The latent curing agent of claim 1, wherein:
the diene includes one or more diene functional groups of the following Chemical Formula 2-1 to Chemical Formula 2-14 or derivatives thereof:

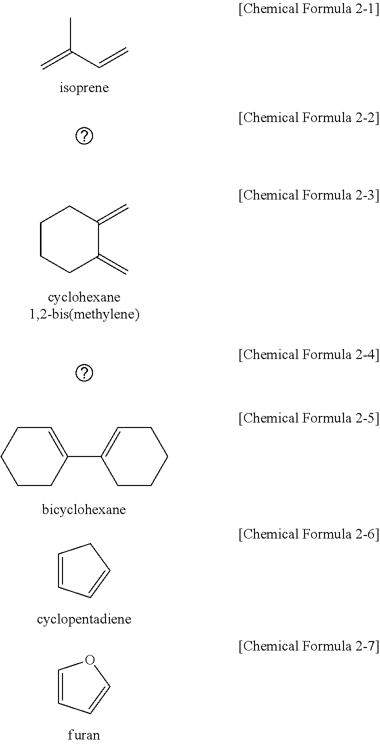

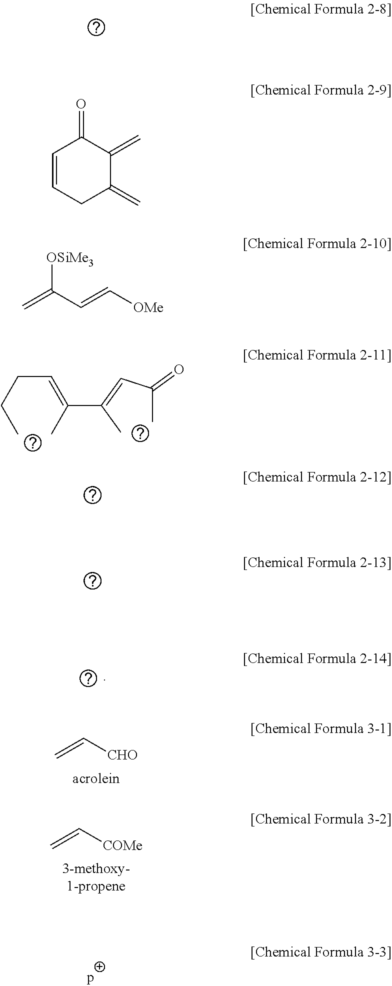

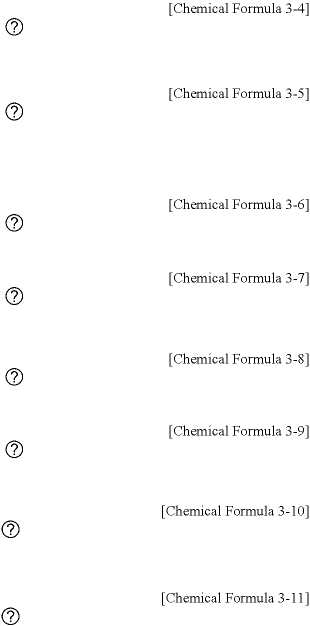

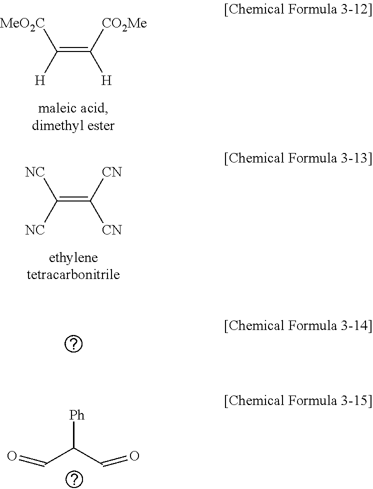 [SMILES: COC(=O)/C=C\C(=O)OC ; N#CC(C#N)=C(C#N)C#N ; O=CC(C=O)c1ccccc1]

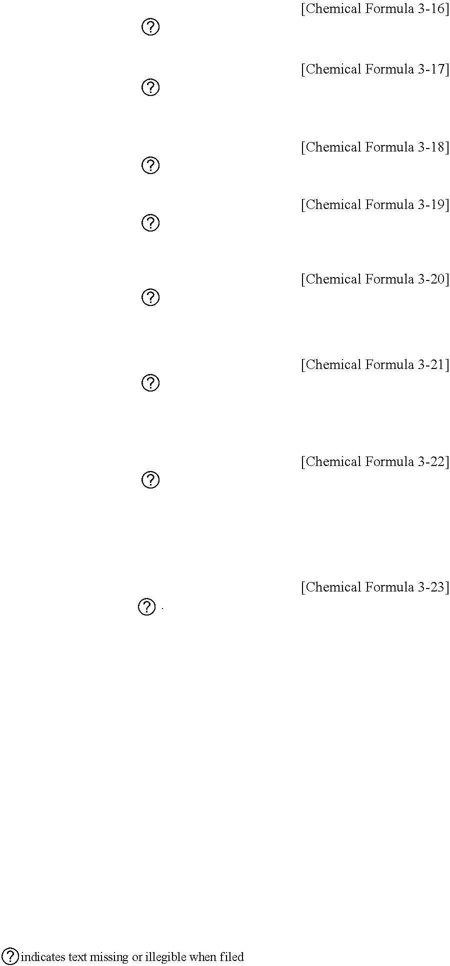

4. The latent curing agent of claim 1, wherein:
the dienophile includes one or more dienophile functional groups of the following Chemical Formula 3-1 to Chemical Formula 3-23 or derivatives thereof:

[symbol]

5. The latent curing agent of claim 1, wherein:
the latent curing agent
starts curing in a temperature range higher than 80° C. and 200° C. or lower.

6. The latent curing agent of claim 1, wherein:
the imidazole-based latent curing agent is
protected by the Diels-Alder reaction between the diene and the dienophile at a temperature in a range of 20° C. to 80° C.

7. The latent curing agent of claim 1, wherein:
the latent curing agent
has an average particle size (D50) in a range of 10 nm to 100 mm.

8. The latent curing agent of claim 1, wherein:
the imidazole-based compound is included in a range of 20 wt % to 90 wt % of the latent curing agent by weight.

9. The latent curing agent of claim 1, wherein:
a diene functional group included in the diene and a dienophile functional group included in the dienophile are included in a range of 0.2:1 to 5:1 based on moles.

10. A method of preparing a latent curing agent, the method comprising:
mixing a diene, a dienophile, and an imidazole-based compound to obtain a first mixed solution;
mixing the first mixed solution with a surfactant to obtain a second mixed solution; and
post-treating the second mixed solution to obtain a latent curing agent,
wherein the obtaining of a second mixed solution is performed in a temperature range of 0° C. to 150° C.

11. The method of preparing a latent curing agent of claim 10, wherein:
the imidazole-based compound is represented by the following Chemical Formula 1: 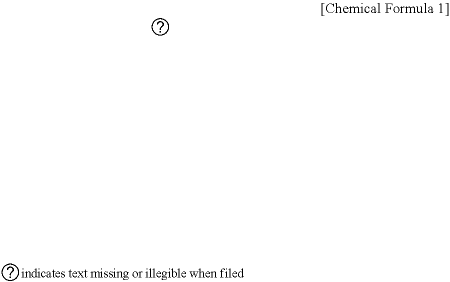
wherein A₁ to A₄ are independently of one another a hydrogen atom, an alkyl group having 1 to 15 carbon atoms, a phenyl group, or a heterocyclic compound.

12. The method of preparing a latent curing agent of claim 10, wherein:
the diene includes one or more diene functional groups of the following Chemical Formula 2-1 to Chemical Formula 2-14 or derivatives thereof:

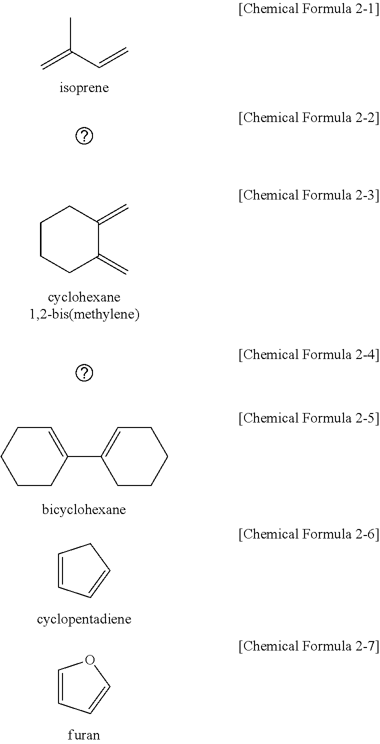

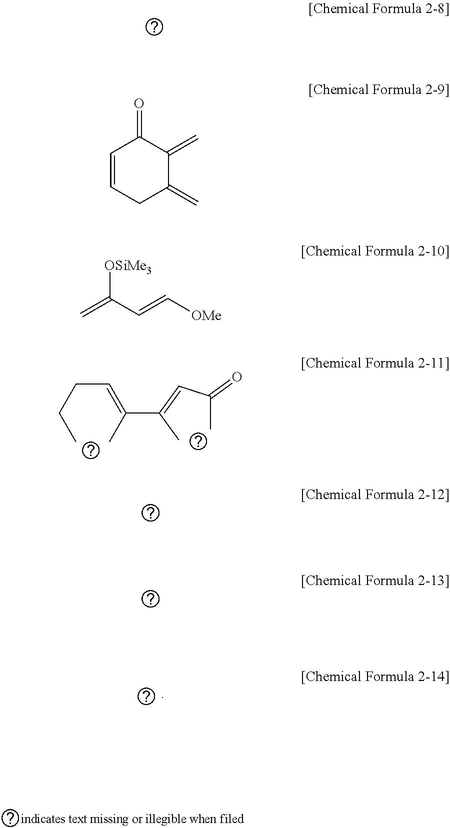

13. The method of preparing a latent curing agent of claim 10, wherein:
the dienophile includes one or more dienophile functional groups of the following Chemical Formula 3-1 to Chemical Formula 3-23 or derivatives thereof:

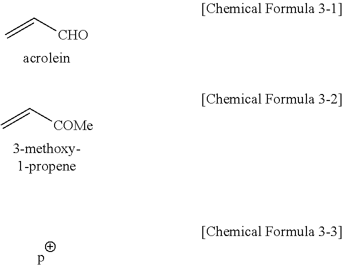

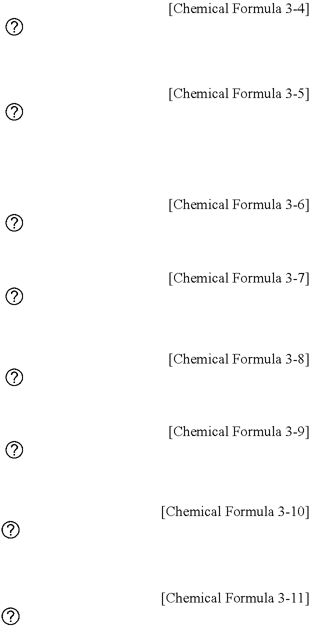

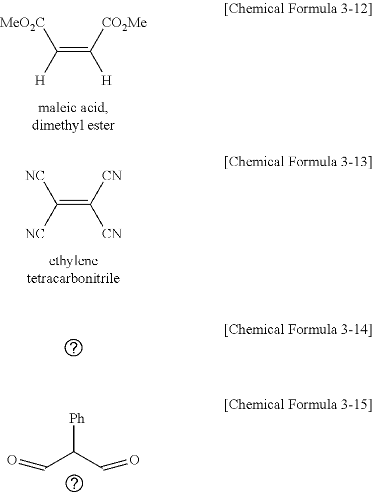 [SMILES: COC(=O)/C=C\C(=O)OC ; N#CC(C#N)=C(C#N)C#N ; O=CC(C=O)c1ccccc1]

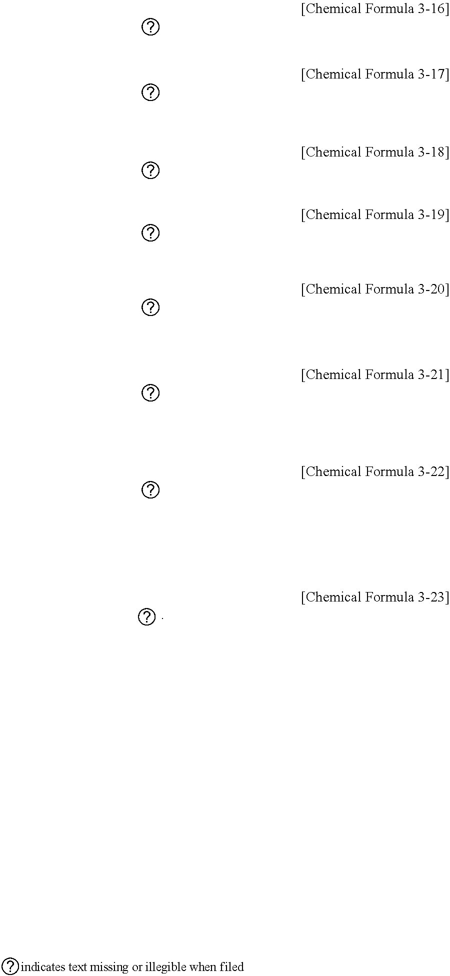

14. The method of preparing a latent curing agent of claim 10, wherein:
the mixing of a diene, a dienophile, and an imidazole-based compound to obtain a first mixed solution is
mixing at a ratio of 0.2:1 to 5:1 based on moles of the diene functional group of the diene and the dienophile functional group of the dienophile.

15. The method of preparing a latent curing agent of claim 10, wherein:
in the mixing of a diene, a dienophile, and an imidazole-based compound to obtain a first mixed solution,
the imidazole-based compound is included in a range of 20 wt % to 90 wt % with respect to a total weight of the diene, the dienophile, and the imidazole-based compound.
